(19) United States
(12) Patent Application Publication
(10) Pub. No.: US 2011/0195962 A1
(43) Pub. Date: Aug. 11, 2011

(54) USE OF MULTI-PHARMACOPHORE COMPOUNDS TO TREAT NASAL DISORDERS

(75) Inventors: Najam A. Sharif, Arlington, TX (US); Martin B. Wax, Westlake, TX (US)

(73) Assignee: ALCON RESEARCH, LTD., Fort Worth, TX (US)

(21) Appl. No.: 13/088,719

(22) Filed: Apr. 18, 2011

Related U.S. Application Data
(62) Division of application No. 12/192,252, filed on Aug. 15, 2008.
(60) Provisional application No. 60/956,223, filed on Aug. 16, 2007.

(51) Int. Cl. A61K 31/54; A61K 31/53; A61K 31/352; A61K 31/4439; A61K 31/506; A61K 31/4164; A61K 31/195; A61K 31/404; A61P 29/00

(52) U.S. Cl. 514/227.5; 514/243; 514/456; 514/341; 514/275; 514/399; 514/561; 514/419

(57) ABSTRACT

Compositions and methods for treating nasal disorders associated with nasal inflammation using a multipharmacophoric agent (MPA) that can prevent the production and/or release of at least one or more pro-inflammatory cytokines (for instance IL-1 and TNFα) and inhibit p³⁸ MAP kinase or at least one matrix metalloproteinases (MMP-1 and MMP-9) are disclosed.

### CROSS-REFERENCE TO RELATED APPLICATION

This application is a divisional of U.S. application Ser. No. 12/192,252 filed Aug. 15, 2008, which claims priority under 35 U.S.C. §119 to U.S. Provisional Patent Application No. 60/956,223 filed Aug. 16, 2007, the entire contents of which are incorporated herein by reference.

### FIELD OF THE INVENTION

The present invention relates generally to methods and compositions to treat nasal inflammatory disorders.

### BACKGROUND OF THE INVENTION

Mucus and fluid hypersecretion is a prominent feature of allergic rhinitis (Carr V. Prof Nurse. 20:31-33, 2005). Biological targets for suppression of hypersecretion range from the inflammatory cells that initiate airway inflammation, to specific cellular elements such as ion channels, growth factors and kinases (Hansen I et al Opin Allergy Clin Immunol. 4:159-163, 2004). Potential approaches to the treatment of allergic rhinitis are the avoidance of allergens and medication with chromone compounds, antihistamines and glucocorticosteroids (Sanico A. M. Clin Rev Allergy Immunol. 27:181-189, 2004). Systemic treatment typically requires higher concentrations of the drug compound to be administered to afford an effective concentration to reach the necessary treatment site. Antihistamine compounds are known to have central nervous system (CNS) activity, which manifests itself in drowsiness. They may also have anticholinergic activity, which manifests itself in the drying of mucus membranes.

Intranasal combination therapy involving anti-allergic agents and steroids is also known. For example, WO 97/01337 discloses combinations of topical nasal antihistamines and topical nasal steroids for the treatment of rhinitis. WO 97/46243 discloses a nasal spray containing a steroid and an antihistamine. β₂ adrenergic receptor agonists are known for use in inhalation products for treating bronchospasm and airway inflammation. At least one combination product containing a β₂ adrenergic receptor agonist and a corticosteroid is commercially available. ADVAIR DISKUS, which contains fluticasone propionate and salmeterol xinafoate, is available from GlaxoSmithKline. No aqueous nasal spray products containing a combination of an anti-allergy agent and a β₂ adrenergic receptor agonist are known.

Nasal mucosal edema can result from irritant chemicals and/or pathogenic agents in the air and such edema can complicate treatment for pulmonary diseases (Ranga and Ackerman, Am. J. Dis. Child. 132: 96, 1978; Berdal, J. Allergy 23: 11-14, 1952; Grudemo, Rhinology 37: 104-107, 1999). Once again, corticosteroids are used to reduce such nasal/pulmonary edema but the long term use of such agents is not recommended and can be complicated further by infections such that new therapeutics agents are needed to address such nasal/pulmonary edematous disorders (Ranga and Ackerman, Am. J. Dis. Child. 132: 96, 1978; Berdal, J. Allergy 23: 11-14, 1952; Grudemo, Rhinology 37: 1040107, 1999).

### SUMMARY OF THE INVENTION

The invention provides methods for treating and/or preventing nasal inflammatory disorder. In certain aspects, a method of the invention comprises administering to a patient a composition comprising a therapeutically effective amount of (a) a multipharmacophoric agent (MPA) possessing inhibitory activities for p³⁸ mitogen-activated protein (p³⁸ MAP) kinase and at least one or more enzymes responsible for synthesis of IL-1 and TNFα, or (b) an MPA possessing inhibitory activities for TNFα and at least one matrix metalloproteinases (MMPs), in a pharmaceutically acceptable vehicle. Specific preferred embodiments of the invention will become evident from the following more detailed description of certain preferred embodiments and the claims. Preferred compounds include FR-167653 (1-{7-4-fluorophenyl)-1,2,3,4-tetrahydro-8-(4-pyridyl)pyrozolo[5,1-c][1,2,4]triazin-2-yl]-2-phenylethanedione sulfate monohydrate; Astellas, Japan), WAY-171318 (TMI-1) (4-[[4-(2-butynyloxy)pheny]sulfonyl]-N-hydroxy-2,2-dimetheyl-(3S)-thiomorpholinecarboxamide; Wyeth, Madison, N.J.); GI5402 (Guilford Pharmaceuticals, Baltimore, Md.); GW3333 (2R,3S)-3-(formly-hydroxy-amino)-2-(2-methyl-1-propyl)-4-methylpentanoic acid [(1S,2S)-2-methyl-1-(2-pyridylcarbamoyl)-1-butyl]amide (Glaxo-Smith-Kline, United Kingdom); Apratastat (TMI-005) (Wyeth, Madison, N.J.); SKF-86002 (6-(4-fluorophenyl)-2,3-dihydro-5-(4-pyridyl)imidazo[2,1-b]thiazole; Calbiochem, San Diego, Calif.); SB-239063 (trans-1-(4-hydroxycyclohexyl)-4-(4-fluorophenyl)-5-(2-methoxypyridine-4-yl)imidazole; Calbiochem, San Diego, Calif.); SB-220025 (5-(2-amino-4-pyrimidinyl)-4-(4-fluorophenyl)-1-(4-piperidinyl)imidazole; Calbiochem, San Diego, Calif.); SB-202190 (4-(4-fluorophenyl)-2-(4-hydroxyphenyl)-5-(4-pyridyl)1H-imidazole; Calbiochem, San Diego, Calif.); ML-3403 ((RS)-{4-[5-(4-fluorophenyl)-2-methylsulfanyl-3H-imidazol-4-yl}-(1-phenylethyl)amine; Calbiochem, San Diego, Calif.); GM-6001 (N-[(2R)-2-(hydroxamidocarbonylmethyl)-4-methylpentanoyl]-L-tryptophan methylamide; Calbiochem, San Diego, Calif.); PD-98059 (2′-Amino-3′-methoxyflavone; Calbiochem, San Diego, Calif.); SB-203580 (4-(4-fluorophenyl)-2-(4-methylsulfinylphenyl)-5-(4-pyridyl)1H-imidazolep; and Calbiochem, San Diego, Calif.).

Specific preferred embodiments of the invention will become evident from the following more detailed description of certain preferred embodiments and the claims.

### DESCRIPTION OF PREFERRED EMBODIMENTS

As discussed above, the inflammatory component of the disease process (signs and symptoms) in rhinitis is now well documented. Some of the major molecular culprits of the etiologies of these disorders involve pro-inflammatory cytokines such as interleukin-1 (IL-1), IL-6, IL-8, TNFα, serum amyloid (SAA), and matrix metalloproteinases (MMPs), and a variety of kinases including p³⁸ mitogen-activated protein (p³⁸ MAP) kinases as described above. Often a single agent is not responsible for mediating the deleterious effects, but additive and/or synergistic effects of the various components drives the disease process and amplifies the inflammatory cascade of events. Accordingly, drugs that inhibit only a single pathway or mechanism are inferior to agents that exhibit multiple therapeutically useful activities, i.e. multivalent or multipharmacophoric agents (MPAs).

In certain embodiments, the invention provides drugs having MPA activities for use in treating nasal disorders. In a particular embodiment, an MPA drug useful in the methods of the invention can inhibit p³⁸ MAP kinase and inhibit (i.e. prevent the production and/or release of) at least one pro-inflammatory cytokine (for instance, IL-1 and TNFα). In another embodiment, an MPA drug useful in the methods of the invention can inhibit at least one pro-inflammatory cytokine (for instance TNFα) and at least one matrix metalloproteinases (for instance, MMP-1 and MMP-9). In a particular embodiment, the MPA can inhibit p³⁸ MAP kinase and both IL-1 and TNFα, or TNFα and an MMP. Such MPA activities are now contemplated to be more useful than singular activity compounds, and are particularly deemed useful, as disclosed herein, for treating nasal diseases using MPA drugs, such as FR-167653, WAY-171318 (TMI-1), GI5402, GW3333, PD-98059, SB-203580, SKF-86002, SB-239063, SB-220025, SB-202190, ML-3403, GM-6001, and apratastat (TMI-005) (Wei et al., Int. Immunopharmacol. 4: 1625-1632, 2004; Zhang et al., J. Pharmacol. Exp. Ther. 309: 348-355, 2004; Conway et al. J. Pharmacol. Exp. Ther. 298: 900-908, 2001; Dekkers et al., Blood, 94: 2252-2258, 1999; Means et al., J. Leukocyt. Biol. 67: 885, 2000). MPA drugs such as these can be used to treat various nasal diseases as described herein, particularly where one or more of inflammatory cytokines (e.g. IL-1, IL-6, IL-8, SAA, or TNFα) and/or MMPs may be elevated due to the disease processes, environmental factors or other chemical/mechanical insults of the nose.

FR-167653 is a potent anti-inflammatory agent that prevents the synthesis of IL-1 and TNFα and possess potent P³⁸-mitogen activated protein kinase (MAPK) inhibitor activity, a key enzyme involved in the production of other inflammatory cytokines (IL-6, IL-8) by TNFα and IL-1 (Yamamoto et al. Eur. J. Pharmacol. 314: 137-142, 1996; Yamamoto et al., Eur. J. Pharmacol. 327: 169-174, 1997; Suzuki et al., FEBS Letts., 465: 23-27, 2000). Furthermore, FR-167653 elevates the levels of the protective cytokine, IL-10 (Wei et al., Int. Immunopharmacol., 4: 1625-1632, 2004) that in turn down-regulates IL-1 and TNFα production (Knoblach and Faden, Exp. Neurol. 153: 143-151, 1998; Bethea et al. J. Neurotrauma 16: 851-863, 1999; Sawad et al. J. Neurochem. 72: 1466-1471, 1999) and which imparts beneficial effects against ischemia/reperfusion injury (Watanabe et al., J. Surg. Res. 101: 146-151, 2001), endotoxin-induced shock (Yamamoto et al., Eur. J. Pharmacol., 327: 169-174, 1997) and prevents fibrosis (Matsuoka et al., Am. J. Physiol. Lung Cell. Mol. Physiol. 283: L103-L112, 2002; Yada et al., J. Thorac. Cardiovasc. Surg. 128: 588-594, 2004). Other useful agents include WAY-171318 (also known as TMI-1) that has multiple useful properties, including ability to inhibit the synthesis of TNFα and MMPs with its utility against rheumatoid arthritis and other inflammation-based diseases (Zhang et al., J. Pharmacol. Exp. Ther. 309: 348-355, 2004). Other useful compounds related to WAY-171318 (TMI-1) include GI5402, GW3333, PD-98059, SB-203580, and Apratastat (TMI-005) (Thabet and Huizinga, Curr. Opin. Invest. Drugs. 7: 1014-1049, 2006).

In certain embodiments, FR167653 and other MPA drugs useful in this invention may contain one or more chiral centers. The present invention contemplates all enantiomers, diastereomers, and mixtures thereof.

In certain embodiments, the invention provides methods of treating nasal disorders, comprising administering an MPA drug to a patient in need thereof. In certain embodiments, the methods of the invention comprise the step of administering a pharmaceutical composition to the nose of a patient, wherein the composition comprises a therapeutically effective amount of an MPA drug and a pharmaceutically acceptable carrier.

The term “nasal disorder” as used herein includes allergic and/or inflammatory conditions of the nose.

In particular embodiments, the invention provides pharmaceutical compositions comprising at least one multipharmacophoric agent (MPA) possessing two or more inhibitor activities, thereby inhibiting the activity of p³⁸ MAP kinase, TNFα-converting enzyme (TACE), interleukin converting enzyme (ICE), MMPs, and/or enzymes releasing IL-1, IL-6 or IL-8.

As used herein, the term “patient” includes human and animal subjects and the term “therapeutically effective amount” refers to the amount of a pharmaceutical composition of the invention determined to produce a therapeutic response in a mammal. Such therapeutically effective amounts are readily ascertained by one of ordinary skill in the art and using methods as described herein.

The terms “pharmaceutical composition” and “composition” as used herein refer to a composition comprising a pharmaceutically acceptable carrier, excipient, or diluent and a MPA as described herein that is capable of inducing a desired therapeutic effect (e.g. reducing or eliminating nasal inflammation) when properly administered to a patient.

In a further embodiment, the nasal compositions are formulated to provide for an intranasal concentration of about 0.1-1000 nM or, in a further embodiment, 1-100 nM. Peak plasma concentrations of up to 20 μM may be achieved for systemic administration. Intranasal compositions are delivered to the nasal mucosa one to four times per day according to the routine discretion of a skilled clinician. The pH of the formulation should range from 4 to 9, or from 4.5 to 7.4. Systemic formulations may contain about 10 mg to 1000 mg, about 10 mg to 500 mg, about 10 mg to 125 mg or 10 mg to 100 mg, for example, of MPA modulating agent. Topical administration directly onto the nasal mucosa via an intranasal insert or implant device or a pharmaceutical drug-delivery-sponge (GELFOAM®, Pharmacia & Upjohn, Kalamazoo, Mich.) may deliver the MPA modulating agent at the rate of 1-2 μl/hour (e.g. 0.0001-10 mg/day) for several weeks according to the device design, its drug release characteristics, and according to the discretion of a skilled clinician.

An “effective amount” means that amount of agent that is able to reduce the symptoms of the nasal disorder under study or the desired end-point. The effective amount of a formulation may depend on factors such as the age, race, and sex of the subject, or the severity of the nasal disorder, for example. In one embodiment, the agent is delivered intranasally at a therapeutic dose thereby ameliorating/reducing the nasal disorder and/or the disease processes.

While the precise regimen is left to the discretion of the clinician, the resulting solution or solutions are preferably administered intranasally as described herein one to four times a day, or as directed by the clinician.

A nasally acceptable carrier refers to those carriers that cause at most, little to no nasal irritation, provide suitable preservation if needed, and deliver one or more agents having MPA modulating activity of the present invention in a homogenous dosage. For nasal delivery, an agent having MPA modulating activity may be combined with nasally acceptable preservatives, co-solvents, surfactants, viscosity enhancers, penetration enhancers, buffers, sodium chloride, or water to form an aqueous, sterile suspension, solution, or viscous or semi-viscous gels or other types of solid or semisolid composition such as an ointment. Nasal solution formulations may be prepared by dissolving the agent in a physiologically acceptable isotonic aqueous buffer. Further, the nasal solution may include a nasally acceptable surfactant to assist in dissolving the agent. Viscosity building compounds, such as hydroxymethyl cellulose, hydroxyethyl cellulose, methylcellulose, or polyvinylpyrrolidone, for example, may be added to the compositions of the present invention to improve the retention of the compound.

In order to prepare a sterile nasal ointment formulation, the agent having MPA modulating activity is combined with a preservative in an appropriate vehicle, such as mineral oil, liquid lanolin, or white petrolatum. Sterile nasal gel formulations may be prepared by suspending the agent in a hydrophilic base prepared from, for example, CARBOPOL®-940 (BF Goodrich, Charlotte, N.C.), or the like, according to methods known in the art for other suitable nasal formulations. VISCOAT® (Alcon Laboratories, Inc., Fort Worth, Tex.) may be used for intranasal injection, for example. Other compositions of the present invention may contain penetration enhancing materials such as CREMOPHOR® (Sigma Aldrich, St. Louis, Mo.) and TWEEN® 80 (polyoxyethylene sorbitan monolaureate, Sigma Aldrich), in the event the agents of the present invention are less penetrating in the nose.

The compounds of the invention can be administered intranasally in the form of a nasal spray or dry powder, as is known to those skilled in the art.

Nasal delivery may be achieved by incorporation of the drug into bioadhesive particulate carriers (<200 μm) such as those comprising cellulose, polyacrylate or polycarbophil, in conjunction with suitable absorption enhancers such as phospholipids or acylcarnitines. Available systems include those developed by DanBiosyst and Scios. The formulation, if in the form of a liquid, can be administered using a simple nasal spray device available from companies such as Valois or Pfeiffer.

When administered in the form of a dry powder, the powdered product can be delivered using an insufflator device, the likes of which are familiar to those skilled in the art. Such devices are manufactured by Teijin (Rhinocort™), Bespak UK, and Valois (Monopoudre™) France.

Other routes of administration include systemically (for example: orally, intravenous, subcutaneous or intramuscular injections; parenterally, dermal or nasal delivery) using techniques well known by those of ordinary skill in the art. It is further contemplated that the agents of the invention may be formulated in intranasal insert or implant devices. For instance, delivery of the MPA modulating agent can be accomplished by endoscopic assisted (including laser-assisted endoscopy to make the incision into the tympanic membrane) injection into the tympanic cavity as set forth, for example, in Amer. J. Otology 16: 158-163, 1995; Ear Nose Throat 76: 674-678, 1997; Otolarngol Head Neck Surg. 120: 649-655, 1999.

Other modes of administration of the MPA modulating agents to treat nasal disorders are via skin patches, intrapulmonary, via liposomes formulated in an optimal manner, and via slow release depot formulations.

MPAs of the present invention may also be readily elucidated by employing the methods and assays described in detail by Fujita et al. Bioorg. Med. Chem. 10: 3113-3122, 2002; Zhang et al. J. Pharmacol. Exp. Ther. 309: 348-355, 2004; Thabet and Huizinga, Curr. Opin. Invest. Drugs. 7: 1014-1049, 2006; Wei et al., Int. Immunopharmacol., 4: 1625-1632, 2004; Watanabe et al., J. Surg. Res. 101: 146-151, 2001; Yamamoto et al., Eur. J. Pharmacol., 327: 169-174, 1997; Matsuoka et al., Am. J. Physiol. Lung Cell. Mol. Physiol. 283: L103-L112, 2002; Yada et al., J. Thorac. Cardiovasc. Surg. 128: 588-594, 2004; Jin et al. Anal. Biochem. 302: 269-275, 2002; Knight et al. FEBS Letts. 296: 263-266, 1992; Beck et al. J. Pharmacol. Exp. Ther. 302: 390-396, 2002; Butler et al. Eur. Cytokine Netw. 6: 225-230, 1995; Knoblach and Faden, Exp. Neurol. 153: 143-151, 1998; Bethea et al. J. Neurotrauma 16: 851-863, 1999; Sawad et al. J. Neurochem. 72: 1466-1471, 1999.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

It should be understood that the foregoing disclosure emphasizes certain specific embodiments of the invention and that all modifications or alternatives equivalent thereto are within the spirit and scope of the invention as set forth in the appended claims.

We claim:

1. A method for treating a nasal disorder, comprising administering to a patient a composition comprising an effective amount of a multipharmacophoric agent (MPA) that can inhibit p³⁸ MAP kinase and prevent production or release of at least one pro-inflammatory cytokine.

2. The method of claim 1, wherein at least one of the pro-inflammatory cytokines is IL-1 or TNFα.

3. The method of claim 1, wherein the MPA can inhibit both IL-1 and TNFα.

4. The method of claim 1, wherein the nasal disorder is associated with nasal inflammation.

5. The method of claim 1, wherein the composition is administered by nasal ointment, intravenous injection, oral administration, intramuscular injection, intraperitoneal injection, transdermal application, transmucosal application, or nasal spray.

6. The method of claim 1, wherein the MPA is FR167653, PD-98059, SB-203580, SKF86002, SB239063, SB22025, SB202190, or ML3403.

7. A method for treating a nasal disorder, comprising administering to a patient a composition comprising an effective amount of a multipharmacophoric agent (MPA) that can inhibit at least one pro-inflammatory cytokine and at least one matrix metalloproteinase.

8. The method of claim 7, wherein at least one of the pro-inflammatory cytokines is TNFα.

9. The method of claim 7, wherein at least one of the matrix metalloproteinases is MMP-1 or MMP-9.

10. The method of claim 7, wherein the nasal disorder is associated with nasal inflammation.

11. The method of claim 7, wherein the composition is administered by nasal ointment, intravenous injection, oral administration, intramuscular injection, intraperitoneal injection, transdermal application, transmucosal application, or nasal spray.

12. The method of claim 7, wherein the MPA is WAY-171318 (TMI-1), GI5402, GW3333, GM6001, or Apratastat (TMI-005).
